(19) United States
(12) Patent Application Publication
(10) Pub. No.: US 2026/0090762 A1
(43) Pub. Date: Apr. 2, 2026

(54) Obstructive Sleep Apnea Episode Detection System

(71) Applicant: Neurostim Technologies LLC, Waltham, MA (US)

(72) Inventor: Hoo-Min D. Toong, Cambridge, MA (US)

(21) Appl. No.: 19/413,014

(22) Filed: Dec. 9, 2025

Related U.S. Application Data
(63) Continuation of application No. 18/307,275, filed on Apr. 26, 2023.
(60) Provisional application No. 63/334,856, filed on Apr. 26, 2022.

(51) Int. Cl. A61B 5/00; A61B 5/08; A61B 5/113; A61B 7/00; A61N 1/36

(52) U.S. Cl. CPC A61B 5/4818; A61B 5/0816; A61B 5/113; A61B 7/003; A61N 1/36031; A61N 1/3606

(57) ABSTRACT

Embodiments detect obstructive sleep apnea (“OSA”) by a user. Embodiments receive audio data emanating from the user while the user is sleeping, the audio data including multiple distinct frequency bands. Embodiments analyze the audio data to detect patterns in frequency content that matches two or more formants of a plurality of formants, the two or more formants indicating a position of a tongue of the user. Based on the analyzing, embodiments determine when the tongue has moved from a forward position to a rear position in an oral cavity of the user. Embodiments generate a signal indicating an OSA episode when it is determined that the tongue has moved from a forward position to a rear position.

### CROSS REFERENCE TO RELATED APPLICATIONS

This application is a continuation of U.S. patent application Ser. No. 18/307,275, filed on Apr. 26, 2023, which claims priority to U.S. Provisional Patent Application Ser. No. 63/334,856 filed on Apr. 26, 2022. The disclosure of each of the foregoing applications is hereby incorporated by reference.

### FIELD

Example invention are directed to systems and methods for improving sleep by detecting the patterns of obstructive sleep apnea episodes.

### BACKGROUND INFORMATION

Obstructive sleep apnea affects the quality of sleep. Obstructive Sleep Apnea (“OSA”) is the intermittent occlusion of the upper airway (“UAW”), resulting in the reduction of airflow through the throat. This may be due to neuromuscular factors or anatomical causes. The muscles that keep the airway open when active can allow it to close when relaxed. An obstructed airflow causes imbalances in oxygen exchange, measurable in the hemoglobin of the blood.

The equipment used for individuals suffering from OSA includes detection of each episode of OSA, in order to apply some treatment to the individual to reduce the effects of such an OSA episode. Detection of an episode occurs once the episode has begun, when the distress of interrupted breathing has begun.

### BRIEF DESCRIPTION OF THE DRAWINGS

FIG. 1 is an Obstructive Sleep Apnea Detection and Stimulation System in accordance to example inventions.

FIGS. 2A-FIG. 2D are diagrams of OSA-Related Nerves and Structures.

FIG. 3 is a User and an Obstructive Sleep Apnea Detection and Stimulation System in accordance to example inventions.

FIG. 4 is a Polysomnography Setup.

FIGS. 5-9 are Summary Graphs from Sleep Apnea Studies.

FIGS. 10-12 are examples of Audio Data Analysis using Formants.

FIG. 13 is a flow diagram of the functionality of the Obstructive Sleep Apnea Detection and Stimulation System for detecting and treating OSA in accordance to example inventions.

Further embodiments, details, advantages, and modifications will become apparent from the following detailed description of the embodiments, which is to be taken in conjunction with the accompanying drawings.

### DETAILED DESCRIPTION

Example inventions includes a system and method that physicians may offer to their patients to detect an OSA episode in preparation for the use of neurostimulation to counteract the apnea, without the complex system of a conventional sleep study in a clinic. Examples apply to both apnea and hypopnea.

Example inventions are directed to an integrated system that is placed on the skin of the User, activated and used with or without the help of a medical professional. The integrated system includes hardware and software to monitor biometrics related to breathing, and optionally, a computing resource able to analyze the breathing data and send improved parametric values to the device on the user's skin to improve the detection capabilities for that specific user, in a closed-loop system. Example inventions include the monitoring of biometrics and the prediction of the onset of an OSA episode.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

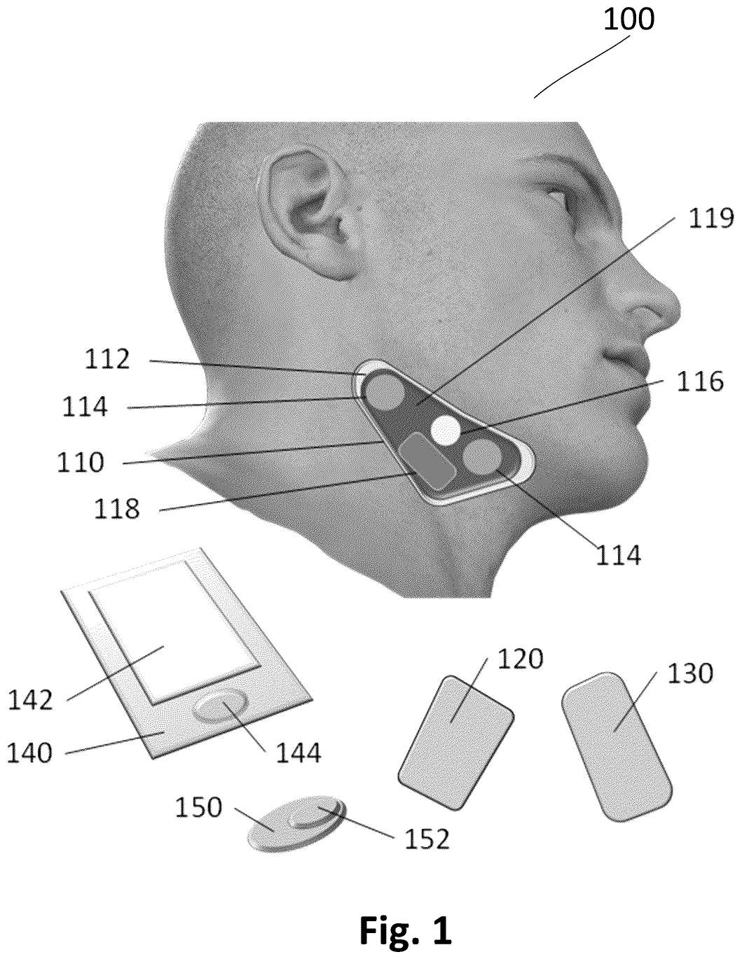

FIG. 1 is an illustration of components of an Obstructive Sleep Apnea Detection and Stimulation System 100, including a Neck Topical Nerve Activator (“TNA”) Device 110 (or “patch” 110), with a securing mechanism 112, and one or more electrode pairs 114 with each pair having a positive electrode and a negative electrode, and a power source 116, and a processor 118; and a Respiration Monitoring Device (“RMD”) 120; and an optional Posture Indication Device 130; and an optional Smart Controller 140, with a display 142 (e.g., a smartphone), and an acknowledgment button 144; and an optional Fob 150 with one or more buttons 152.

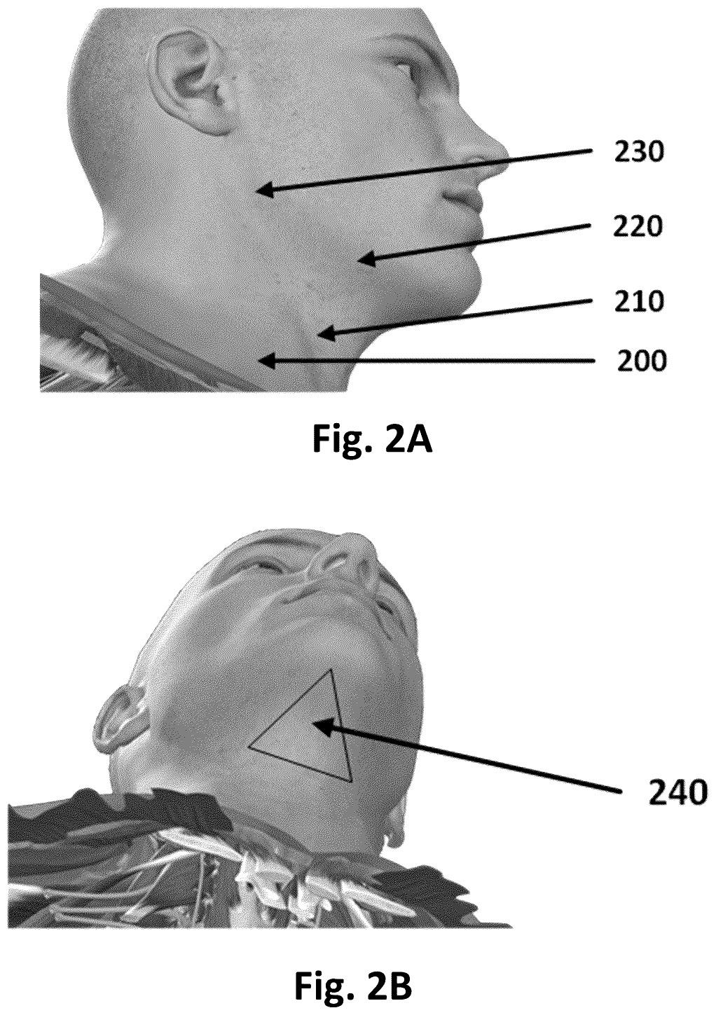

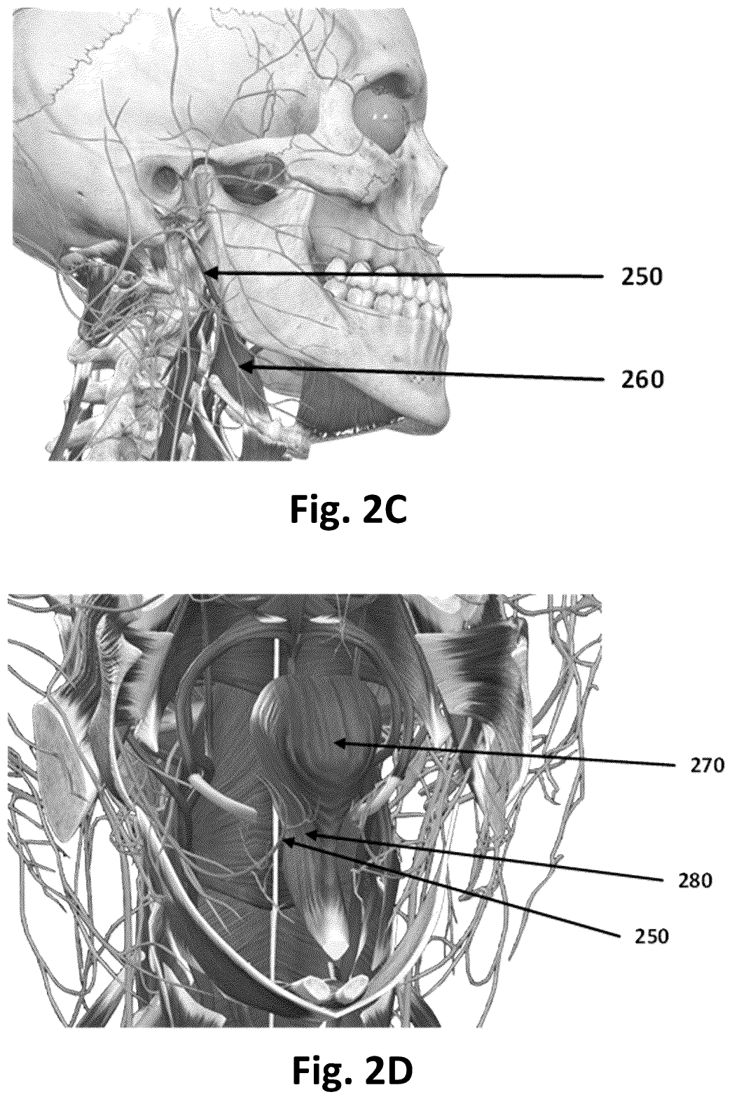

FIG. 2A shows a User 200, with a Neck 210, a Jaw Line 220, and an area behind the mandible 230.

FIG. 2B shows the User 200 and the submental triangle 240 below the chin.

FIG. 2C shows the User 200 and an internal view of the hypoglossal nerve 250 and the sublingual nerve 260.

FIG. 2D shows the User 200 and an internal view of the hypoglossal nerve 250, and the genioglossus muscle 280 under the tongue 270.

In FIG. 1, the Neck TNA Device 110 is in a shape to conform to a selected dermis surface to be electronically effective at stimulating the hypoglossal nerve 250 and to monitoring breathing. The Neck TNA Device 110 can be used for apnea detection as well as for delivering electrical stimulation. The Neck TNA Device 110 is electronically most effective for stimulation when the positive and negative electrodes are placed axially along the path of the nerve in contrast to transversely across the path of the nerve, which is not as electronically effective.

The Neck TNA Device 110 shape is designed to minimize discomfort for the User 200 when affixed in the target location.

The Neck TNA Device 110 includes one or more sensors that measure internal features or biometrics of the User in the neck area. These measurements are used to orient and place the Neck TNA Device most accurately in the target location and to monitor biometrics related to breathing. The sensor data is communicated to one or more of the Smart Controller 140, and the Fob 150; and is used by the Neck TNA Device.

In some examples, an indication such as LED or vibration is sent to the User to assist them in placing the Neck TNA Device.

TNA Device/patch 110 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 110 in one example provides topical nerve or tissue activation/stimulation on the user to provide benefits to the user, including treatment for OSA.

Patch 110 in one example can include a flexible substrate, a malleable dermis conforming bottom surface of the substrate including adhesive and adapted to contact the dermis, a flexible top outer surface of the substrate approximately parallel to the bottom surface, a plurality of electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip or discrete components and including an electrical signal generator integral to the malleable dermis conforming bottom surface configured to electrically activate the one or more electrodes, a signal activator coupled to the electrical signal generator, a nerve/tissue stimulation sensor that provides feedback in response to a stimulation of one or more nerves or tissues, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves or tissues of a user wearing patch 110 at least at one location proximate to patch 110. Additional details of examples of patch 110 beyond the novel details disclosed herein are disclosed in U.S. Pat. No. 10,016,600, entitled “Topical Neurological Stimulation”, the disclosure of which is hereby incorporated by reference.

FIGS. 2A and 2B show how the Neck TNA Device 110 is designed to be placed on either side of the neck, or on the jaw in one of several locations, such as the submental triangle 240, below the jawline 220, or behind the mandible 230; situated to accurately monitor biometrics related to breathing.

### Detection Using Respiration Monitoring Device

The function of the Respiration Monitoring Device (“RMD”) 120 is to detect occurrences of interrupted breathing due to an apnea episode or hypopnea episode, and to notify one or more of the Neck TNA Device 110, or the Smart Controller 140, or the Fob 150 of such an episode.

OSA has traditionally been diagnosed through a sleep study, or polysomnography, usually performed in a clinic setting with multiple electrodes monitoring multiple body parameters. Requests for home screening tests are rising because of comfort and cost issues. The STOPBANG scale has been used to provide guidance to those individuals assumed to be at high risk for moderate to severe OSA, with the acronym defined as Snoring; Tiredness in daytime; Observation by third party of stopped breathing; high blood Pressure; Body Mass Index (Bmi) greater than 35; Age over 50; Neck circumference greater than 16 inches; and male Gender. A STOPBANG score of 3 or more indicates a home test or sleep study should be performed, especially since up to 80% of people with OSA do not know they have apnea.

The RMD 120 includes one or more of accelerometers and an audio sensing device. The accelerometer detects rhythmic movements of respiration, or the lack of such movement. The audio sensing device detects the sounds of air flowing through the throat, snoring, body motion in bed, and background noise.

The RMD 120 captures data from one or more of its accelerometers and audio sensing device as a time-based series of measurements, and sends the data the Smart Controller 140, which modifies the data series to derive the power signature of movement data from the accelerometers and the power signature of audio data from the audio sensors. By filtering the data to remove power components outside the frequencies which are indicative of inspiration and expiration, the Smart Controller deduces the series of breaths in the data series.

An example, the Smart Controller 140 measures the frequency content across multiple distinct frequency bands in a continuous manner, recording that stream of measurement data in its memory. The data is analyzed in real-time to detect patterns of frequency content that match two or more formants, similar to formants characteristic of human speech. The formants are chosen from the full set of speech formants to use those which indicate tongue position. By matching the real-time signature in the breathing audio to the signatures of the formants, the Smart Controller marks times when the tongue moves from a forward position to a rear position in the oral cavity. When the Smart Controller detects that the tongue has moved or is moving into the airway, the Smart Controller signals to the Neck TNA Device 110 that an apnea episode has begun.

An example, the software converts the time domain audio signal into the frequency domain, such as by fast Fourier transform or digital Fourier transform or similar means, and creates a data sequence to show, at each sampled time step, the energy content of the audio at each specified frequency band.

An example, the frequency bands are determined at the beginning of the device usage from a default set.

An example, the frequency bands are determined at the beginning of the device usage during a training period, during which the device monitors the user and calculates the formant content for that specific user; followed by the diagnostic period, in which the audio data is distilled into formant energies according to the previously-determined formant definitions.

Following the previous example, the frequency bands continue to be adjusted during the diagnostic period, thereby using a closed-loop system to improve the distillation of apnea-related energies based on the real-time changes in the user's breathing, such as when the user changes sleeping positions, which may be detected by motion sensors on the device, or when the user's nasal or oral passageway changes as in congestion.

In some examples, the formant recognition is performed by firmware or software in the Neck TNA Device 110.

In some examples, the formant recognition is performed by firmware or software in the RMD 120.

In some examples the duration of each inspiration is measured by the RMD 120 to estimate the magnitude of the indrawn breath, the Obstructive Sleep Apnea Detection and Stimulation System 100 using this measurement to distinguish apnea events from hypopnea events. Thus, shorter, incomplete breaths will have a duration distinguishable from longer, complete breaths, using a threshold established by the Obstructive Sleep Apnea Detection and Stimulation System 100 or set and adjusted periodically by the Smart Controller 140, based on filtering of data to discern a minimum indrawn breath indication for the specific user.

The RMD 120 can be positioned at various locations on the body, depending upon which sensor and which body parameters are measured. For example, an RMD containing an accelerometer is positioned on the chest to detect rhythmic breathing patterns or on the neck in the submental region, or on the lower neck at the suprasternal notch; whereas an RMD containing an audio sensor can be positioned near the outside of the nasal passageway or on the neck in the submental region, or on the lower neck at the suprasternal notch.

In some examples, the RMD 120 is a separate unit from the Neck TNA Device 110, is positioned at various anatomical locations around the body away from the Neck TNA Device.

In some examples the RMD 120 is integrated within the Neck TNA Device 110, and monitors specific body signals at the same location as the Neck TNA Device.

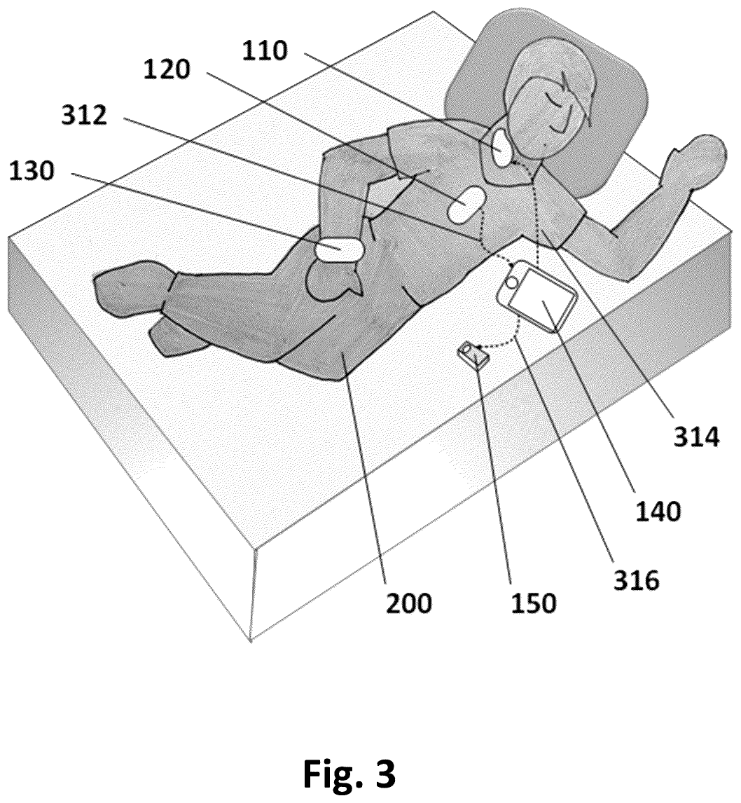

FIG. 3 illustrates an Obstructive Sleep Apnea Detection and Stimulation System 300 that includes a Neck TNA Device 110, and a Respiration Monitoring Device (“RMD”) 120. The RMD may be a separate unit or integrated within the Neck TNA. It may also include a Smart Controller 140 or Fob 150. The User 200 may indicate to the Neck TNA Device or Smart Controller or the Fob directly when the User is beginning a Sleep Period and again when the User is ending a Sleep Period. During the Sleep Period, when the RMD senses an OSA episode, the RMD then signals the Smart Controller or Fob that the apnea be recorded and optionally suppressed using the Neck TNA Device, then the Smart Controller signals to the Neck TNA Device to activate the nerve, then the Smart Controller optionally signals to the Fob to record such an activation event that the apnea is suppressed for a period of time.

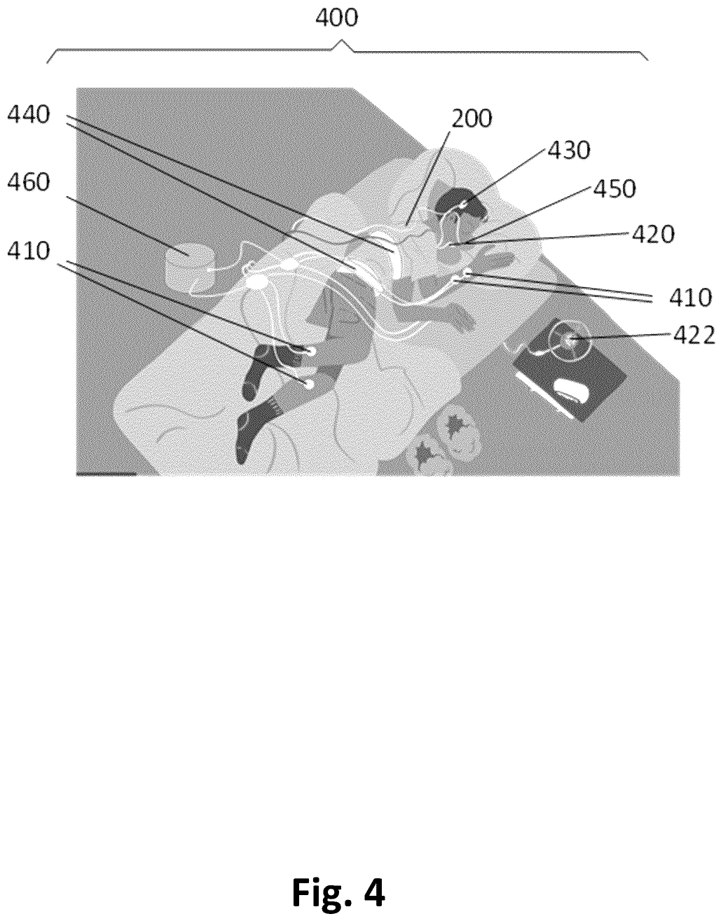

FIG. 4 illustrates a conventional polysomnography (“PSG”) system 400 that may include a number of sensors and wires attached to the User 200 while sleeping, including a set of Leg Movement Sensors 410 with wires, a Snoring Sensor Microphone 420, an Ambient Noise Microphone 422, an EEG Electrode 430, one or more Breathing Detection Belts 440, and an Airway Sensor 450. As may be seen in the figure, the wires connecting the sensors to the PSG Controller 460 may interfere with the User's sleep, or the measurements may be adversely affected by User movement, or the measurements may be stopped due to disconnection of one or more sensor due to User movement.

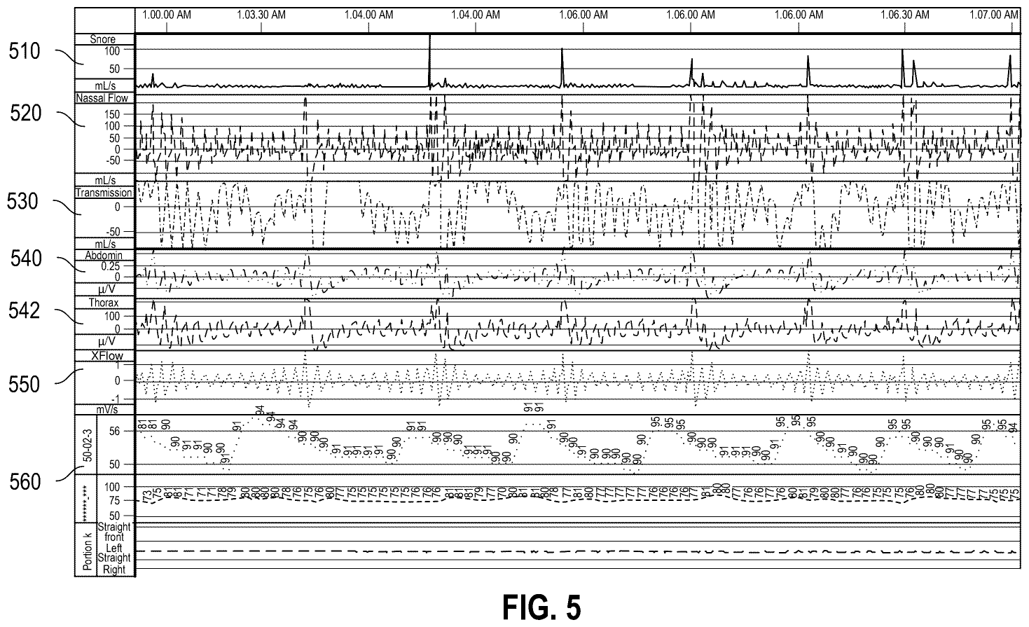

FIG. 5 illustrates an example of signals captured during a sleep period. Snoring is detected along Snoring Signal 510; Breathing is detected along Nasal Flow Signal 520, and Thermistor Signal 530; User's movement is detected along Abdomen Signal 530, and Thorax Signal 540; respiratory airflow volume is detected along XFlow Signal 550, and SpO2 Oxygen Saturation Signal 560.

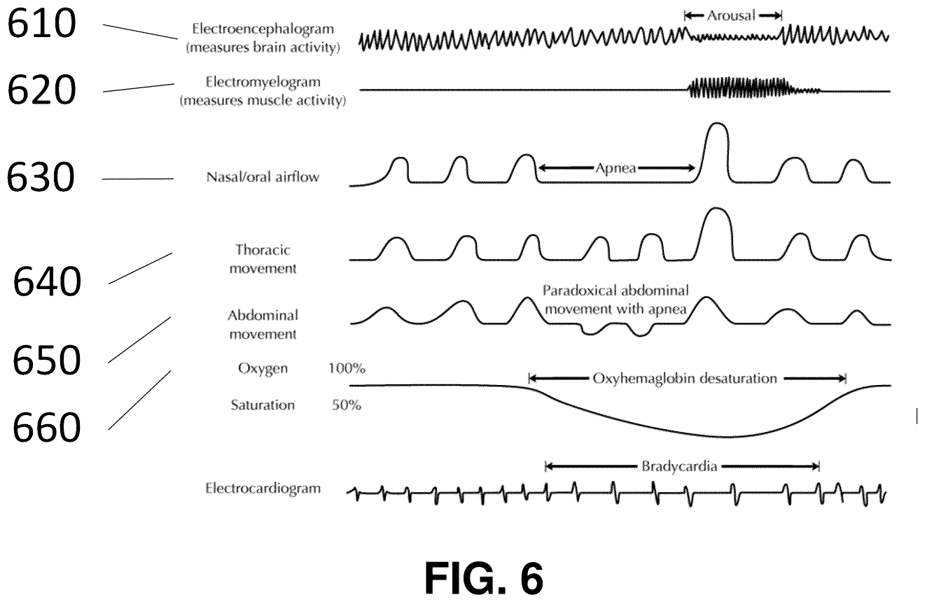

FIG. 6 summarizes measurements showing biometric data during a sleep study. The graph shows how only the Nasal/Oral Airflow Signal 630 indicates a sleep apnea event without trailing the sleep apnea event, such as is shown by the Oxygen Saturation Signal 660 and the EMG Signal 620 which are trailing signals. The other signals: the EEG 610, the Thoracic Movement Signal 640, and the Abdominal Movement Signal 650—do not distinctively indicate a sleep apnea event.

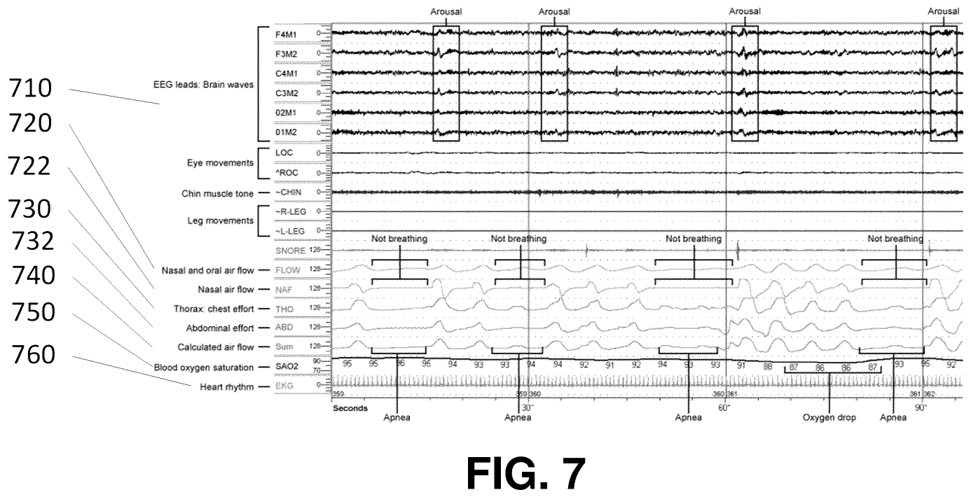

FIG. 7 summarizes measurements showing biometric data during a sleep study. The graph shows how airflow, measured by the Nasal and Oral Air Flow Signal 720, the Nasal Air Flow Signal 722, the Thorax Chest Effort 730, the Abdominal Effort 732, and the Calculated Air Flow 740 all indicate a sleep apnea event without trailing the sleep apnea event as is shown by the EEG Signal 710. The other signals: the Blood Oxygen Saturation Signal 750, and the Heart Rhythm Signal 760—do not distinctively indicate a sleep apnea event.

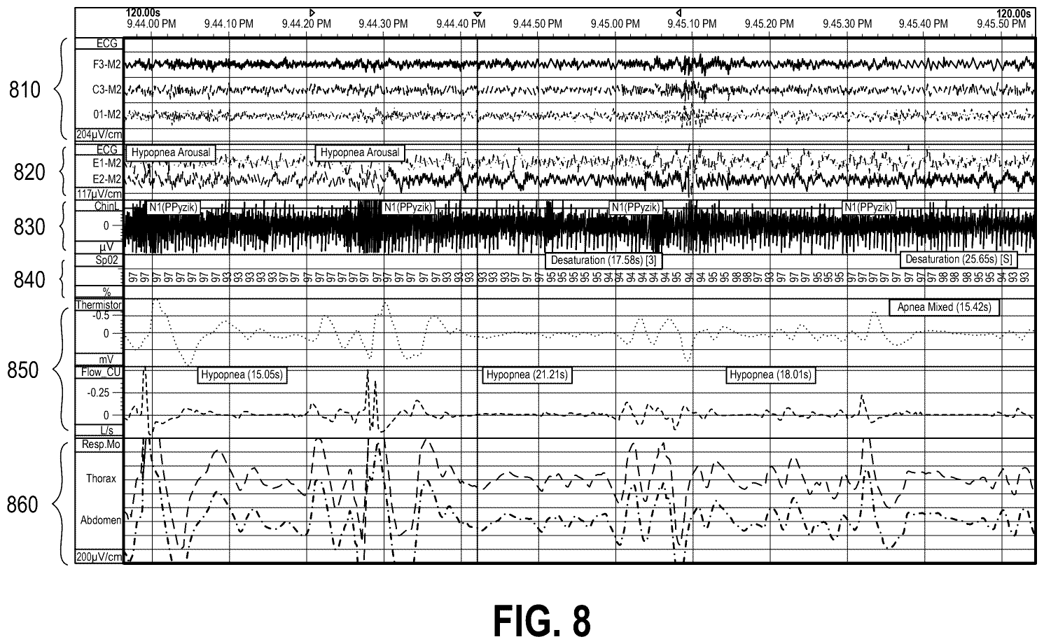

FIG. 8 summarizes measurements showing biometric data during a sleep study. Apnea events are strongly indicated coincident by Breathing Airflow Measurements 850, and somewhat indicated coincident by Breathing Effort Measurements 860; but either not clearly indicated or only indicated after the onset of a sleep apnea event by EEG Measurements 810, Electroculogram (“EOG”) Measurements 820, Electromyelogram Measurements 830, and Pulse Oximetry Measurements 840.

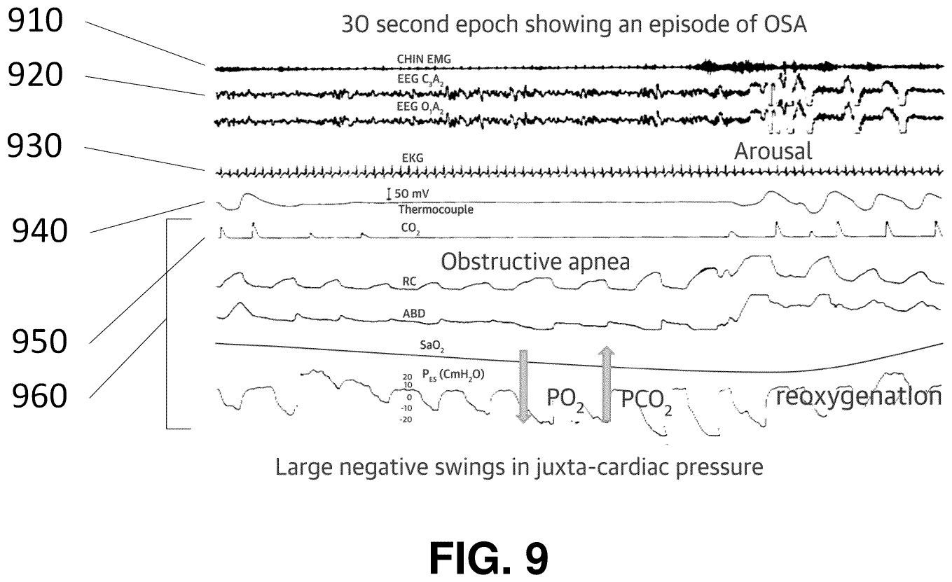

FIG. 9 summarizes measurements showing biometric data during a sleep study. Apnea events are only indicated after the event by EMG Measurements 910, EEG Measurements 920, EKG Measurements 930, and Blood Chemistry Measurements 960. The Thermocouple Measurements 940, from which air flow is calculated, and CO2 levels 950 correlate with coincident timing to sleep apnea events.

The graphs shown in FIG. 5 through FIG. 9 demonstrate that a measurement of breathing air flow is sufficient to indicate a sleep apnea event at a time coincident with the event and not trailing in time after the event has begun.

### Audio Data Analysis

An example, the audio stream collected by the one or more microphones in the TNA Device 110 is a digital sequence of values, sampled in time at a sampling frequency adequate to represent frequency content of the audio up to the maximum frequency of interest (“FOI”). Software converts the amplitude data in the time domain into a time sequence of frequency energy levels, each of a set of frequency domain values pertaining to the energy in a defined frequency range, including the FOI. In examples, the frequency data is analyzed with a set of formant amplitudes.

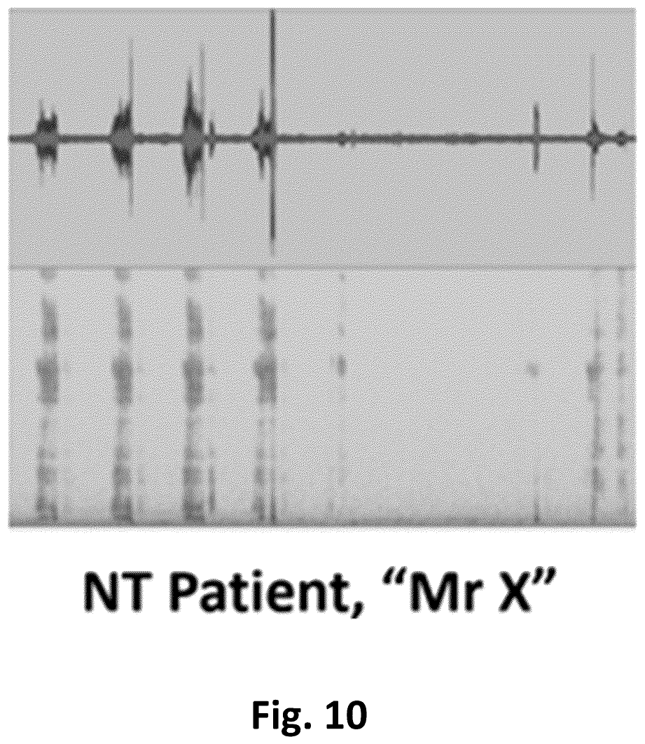

FIG. 10 illustrates an example of audio data in the time domain converted to a time-series of frequency energy content. The time axis is on the horizontal in both graphs. The top graph shows the audio signal as an amplitude measured by one or more microphones. The top graph includes peaks of sound during each breath, with relative quiet between breaths. The bottom graph shows frequency content, with frequency along a range from low to high on the vertical axis, and intensity of energy indicated by color of the plot at each time point. Thus, in the bottom graph, the darker spots indicate higher energy at the frequency read out on the vertical axis.

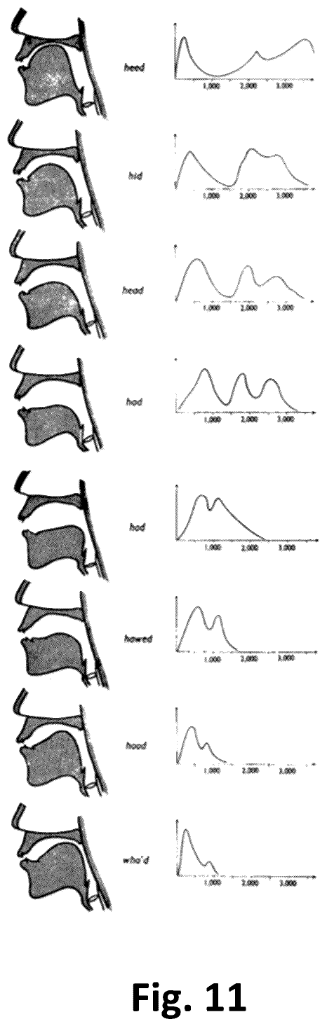

FIG. 11 illustrates the type of formants associated with snoring and breathing during sleep. It also illustrates the position of the tongue during these formant characteristics.

Formants are specific speech constructs that are used to identify spoken speech patterns, which can include vowels, fricatives, stops, full words, connected sentences and confounding factors such as intonation, accents, and emphasis. Formants are the peaks of the sound spectrum that are produced by the vocal tract when someone speaks. The vocal tract includes the mouth, throat, and nasal passages, and its shape and size determine the frequencies of the formants.

Each vowel sound has a characteristic pattern of formants, which allows us to distinguish between different vowels. For example, the first formant, or F1, is usually lower in frequency for back vowels like “ah” (as in “father”) and higher for front vowels like “ee” (as in “feet”). The second formant, or F2, is higher for front vowels and lower for back vowels. These differences in formant patterns allow us to recognize and distinguish between different vowel sounds.

Formants are also important for speech perception because they convey information about the speaker's vocal tract, such as their gender, age, and emotional state.

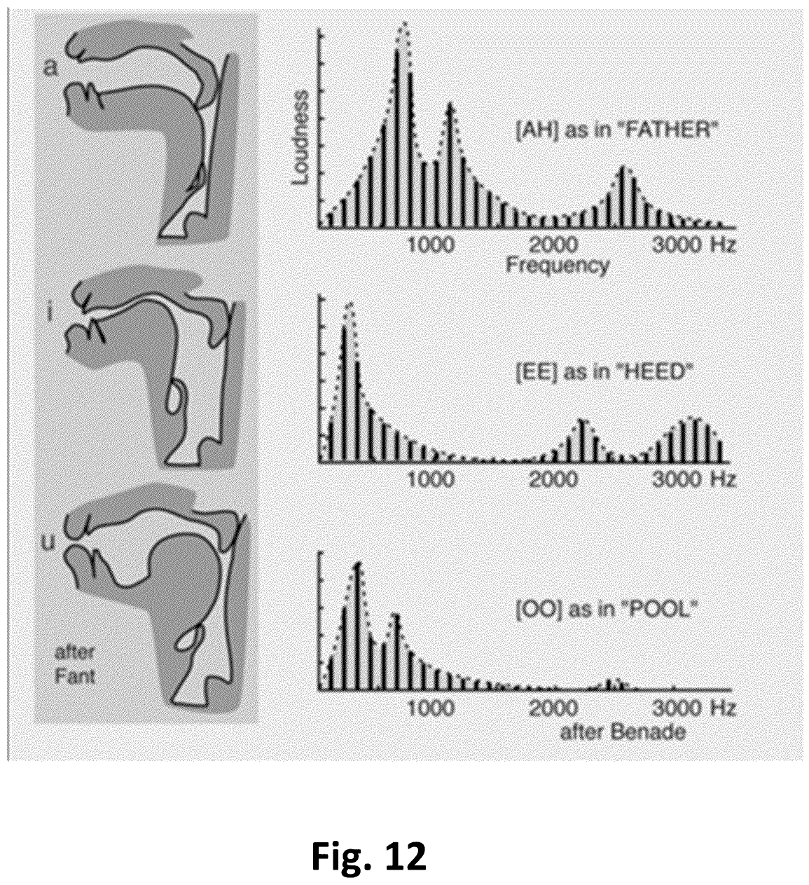

FIG. 12 illustrates the formants associated with vowel sounds and shows specific examples of tongue positioning in the oral cavity and the volume associated with the pharyngal cavity.

OSA is caused by the tongue moving/falling back to the rear of the oral cavity to the point of blocking the pharynx, or air flow.

In English, as in other languages, each vowel sound is formed with a particular set of formants. Each formant is composed of multiple fundamental frequencies of audio energy, designated F1, F2, F3, etc. The largest distinctions between vowel sounds are in F1 and F2.

Note that the formant frequency difference between F1 and F2 narrows with the vowel sounds where the tongue is towards the back of the oral cavity. The vowel sounds incorporating a “oo”” as in “pool” or “good” have the smallest differences and the lowest set of frequencies.

From the field of speech analysis and the relationship of vowel articulation and tongue position to spectral representation of F1/F2, a correlation is established between tongue position in the oral cavity and the first two formant frequencies, F1 and F2. As the frequency distance between F1 and F2 decreases, and the overall frequency measurements of F1 and F2 decrease in real time, the system can infer that the tongue is starting to move towards the back of the oral cavity, such a change potentially signaling the onset of an OSA event.

The spectral plots of the breathing signal show the F1/F2 formants.

By extracting accurate formant data of F1/F2 in time, the system passes the formant data values on to either an analytical detection model or a machine learning model. The machine learning model trains and detects OSA events based mainly on the pattern of F1/F2 evolution over time.

An example, the formant data couples with other spectral data, such as “dead spots”of minimal spectral energy.

Analytic models use the accurate F1/F2 data to detect the onset of an OSA event, combined with the additional data of zero/minimal spectral energy. The accuracy of this prediction improves when coupled to the machine learning model where the F1/F2 indication of onset, coupled with following zero/minimal spectral energy will exemplify a “positive” training instance, and many such “positive” examples are used to train the machine learning model.

In an example, the system uses a closed-loop feedback flow to analyze the F1/F2 data with a machine learning model, then sends that analysis back to change and improve the recording and analysis parameters, such as those used in the machine learning model. For example, machine learning can be used to detect patterns of formants or breathing to permit the real time treatment of OSA and to adjust the treatment over time as the patterns of formants or breathing changes (e.g., parameters relating to the movement of the tongue, such as the rate of movement, duration of the tongue remaining in a position in the mouth, etc.).

In examples, when an OSA event is detected, patch 110 generates electrical charges on the electrodes to initiate an OSA treatment by stimulating the genioglossus muscle, with the hypoglossal nerve, with the result being causing unconscious movement of the user's tongue, and “treating” the sleep apnea condition. In other examples, other nerves can be stimulated that cause movement of the tongue through a reflex (i.e., a rapid, involuntary response to a stimulus) and/or a reflex arc (i.e., the pathway traveled by the nerve impulses during a reflex). For example, the following reflexes cause the tongue to move in response to electrical stimulation:
Glossopharyngeal/Hypoglossal reflex: Stimulation of the glossopharyngeal and superior laryngeal nerves results in the excitation of the genioglossus muscles. This reflex results in a protrusion of the tongue.
Jaw/tongue reflexes: Opening of the jaw results in reflexive activity of the genioglossus muscle.
Masseter Reflex: Stimulation of masseter afferents excites muscles which protrude the tongue, (primarily the genioglossus) and inhibits opposing muscles.
Temporalis Reflex: Light pressure on temporalis muscle activates hypoglossal motoneurons that innervate the tongue muscles.
Pharyngeal Reflex: Sensory mechanoreceptors located within the pharyngeal mucosa play an important role in the reflex which maintains upper airway patency. For example, pharyngeal negative pressure sensors activate dilatory muscles of the upper airway and help stabilize the airway.

### Second Person

In some examples, the Smart Controller 140, or the Fob 150, or both, is in control of a sleep partner or medical service provider such as a sleep researcher. When the Respiration Monitoring Device 120 detects an apnea episode, a notification is sent to the Smart Controller, or to the Fob, whereupon the second person may record the apnea episode.

In some examples, the second person may activate the stimulator based upon visual and auditory clues arising from the sleeping individual. The second person can also observe the effects of stimulation upon the User, and record reactions, either electronically in the Smart Controller 140 or in the Fob 150, or manually such as in a sleep diary.

In some examples, the second person may monitor and respond to the signals from multiple Users'Obstructive Sleep Apnea Detection and Stimulation Systems, or from a database of historical recordings of the User's sleep patterns, or a database of a large population of anonymized User sleep recordings that have been analyzed with pattern recognition or AI techniques including machine learning and deep learning techniques.

In some examples, the Obstructive Sleep Apnea Detection and Stimulation System 100 may use electrocardiogram (“ECG”), or encephalogram (“EEG”), or other means to detect the User in the state of rapid eye movement (“REM”) sleep, or in non-REM sleep; and the system may apply apnea treatments in a manner appropriate to each type of sleep.

### Configurations

In some examples, the Respiration Monitoring Device 120 may signal directly to the Neck TNA Device 110 to suppress the apnea episode, bypassing the Smart Controller 140.

The Neck TNA Device 110, the Respiration Monitoring Device 120, the Posture Indication Device 130, the Smart Controller 140, and the Fob 150 may be combined in a variety of ways to implement an Obstructive Sleep Apnea Detection and Stimulation System 100.

In some examples, the User 200 uses the Fob 150 to send data and controls to the Smart Controller 140.

In some examples, the User 200 uses the Fob 150 to send data and controls to the Neck TNA Device 110.

In some examples, the User 200 uses the Smart Controller 140 directly, and a Fob 150 is not used.

In some examples, the Fob 150 communicates data and controls with the Smart Controller 140 or to the Neck TNA Device 110, or both, through wireless means.

In some examples, the User 200 does not wear the Neck TNA Device 110, or the Respiration Monitoring Device 130, or both, when in the non-prone or waking state.

In some examples, one or both of the Neck TNA Device 110 and the Smart Controller 140 perform analysis of the Respiration Monitoring Device 120 measurements and the Posture Indication Device 130 measurements.

In some examples, the communication of data and control among the Smart Controller 140, Neck TNA Device 110, Respiration Monitoring Device 120, and Posture Indicator Device 130 may be by wireless means through the use of Bluetooth Low Energy (“BLE”), Wi-Fi, or other means.

In some examples, the Respiration Monitoring Device 120 and the Posture Indicator Device 130 may be combined into one unit with a common processor and common power source, data and control between the Respiration Monitoring Device and the Posture Indicator Device being in this case through wired or wireless means. This combined unit may communicate data and control with the Smart Controller 140 and the Neck TNA Device 110 through wireless means.

In some examples, the Respiration Monitoring Device 120 and the Posture Indicator Device 130 and the Smart Controller 140 may be combined into one unit with a common processor and common power source, data and control between the Respiration Monitoring Device and the Posture Indicator Device and the Smart Controller being in this case through wired or wireless means. This combined unit may communicate data and control with the Neck TNA Device 110 through wireless means.

In some examples, the Respiration Monitoring Device 120 and the Posture Indicator Device 130, the Smart Controller 140, and the Neck TNA Device 110 may be combined into one unit with a common processor and common power source, data and control between the Respiration Monitoring Device and the Posture Indicator Device and the Smart Controller and the Neck TNA Device 110 being in this case through wired means.

The Neck TNA Device power source 116, the Respiration Monitoring Device 120, the Posture Indicator Device 130, and the Smart Controller 140, and the Fob 150 may be powered by battery or rechargeable means.

### Data Collection

In some examples, analysis of measurements from one or both of the Smart Controller 120 and the Respiration Monitoring Device 120 may be performed by processing in a remote server, in the cloud, or on a computer separate from the Smart Controller but local to the User, such as a personal computer.

In some examples, the Obstructive Sleep Apnea Detection and Stimulation System 100 measures the User's sleep schedule over a period of days or weeks or longer, noting the clock time when the User begins the sleep period and the clock time when the User wakes during or at the end of the sleep period. The system analyzes this data and determines the most effective clock times to activate the Obstructive Sleep Apnea Detection and Stimulation System.

In some examples, Obstructive Sleep Apnea Detection and Stimulation System 100 collects time-based records of a User's sleep. These records are entered into a database of anonymized sleep period information from large populations of Obstructive Sleep Apnea Detection and Stimulation System Users, or with recordings of sleep periods from other detection systems.

In some examples, the OSA Detection System 100 uses AI techniques such as pattern recognition and correlation analysis to correlate real-time data recordings of the User with larger population databases to produce comparative or predictive analyses. In some examples, machine-learning algorithms are employed to build up the User's sleep history and provide specific predictors of sleep apnea severity and associated conditions.

In some examples, the time-based records of sleep periods are supplemented with data entered manually by one or more observer of the User's sleep.

The data recorded in the time-based database is sent to the cloud through a local network, such as a home mesh network, or directly over the Internet.

### Use of OSA Detection System for Sleep Studies

Sleep studies that employ polysomnography are considered the gold standard of tests used to diagnose sleep disorders. Polysomnography monitors brain waves (“EEG”), blood oxygen levels, heart rate, breathing, and eye and leg movements. These tests are generally conducted in sleep clinics and require overnight observation by trained sleep technicians as well as physicians. During the sleep study, if apnea is observed, the patient is awakened and a CPAP device is applied to the patient to continue to observe and record the same biometric signals while under the CPAP treatment.

Using the Obstructive Sleep Apnea Detection and Stimulation System 100, when an apnea event is observed by the system, stimulation can immediately be provided by the system and monitored through the RMD 120. Use of the Obstructive Sleep Apnea Detection and Stimulation System avoids awakening the User 200 to administer CPAP to observe the effects of treatment. Thus, a sleep study with recording of basic OSA signals such as with the RMD can be done at home without the artifacts possibly introduced by a sleep clinic.

The convenient and comfortable use of the Obstructive Sleep Apnea Detection and Stimulation System 100 by the User 200 allows the system to collect data over a longer period of time without undue interference with sleep or other inconvenience when compared to conventional PSG systems 400.

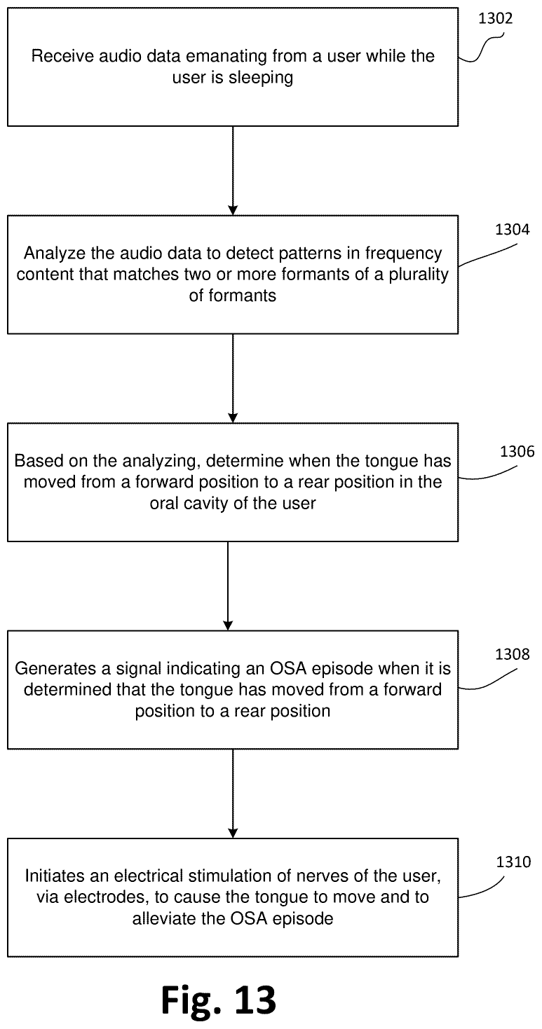

FIG. 13 is a flow diagram of the functionality of the Obstructive Sleep Apnea Detection and Stimulation System 100 for detecting and treating OSA in accordance to example inventions. In one example, the functionality of the flow diagram of FIG. 13, is implemented by software stored in memory or other computer readable or tangible medium, and executed by a processor. In other examples, the functionality may be performed by hardware (e.g., through the use of an application-specific integrated circuit (“ASIC”), a programmable gate array (“PGA”), a field programmable gate array (“FPGA”), etc.), or any combination of hardware and software. The processor can be the processor on patch 110, a processor in an external device in communication with patch 110 (e.g., smart controller 140), or a combination.

At 1302, the system receives audio data emanating from a user while the user is sleeping. The audio data includes multiple distinct frequency bands.

At 1304, the system analyzes the audio data to detect patterns in frequency content that matches two or more formants of a plurality of formants, where the two or more formants indicate a position of the tongue of the user.

At 1306, based on the analyzing, the system determines when the tongue has moved from a forward position to a rear position in the oral cavity of the user.

At 1308, the system generates a signal indicating an OSA episode/event when it is determined that the tongue has moved from a forward position to a rear position.

At 1310, the system initiates an electrical stimulation of nerves of the user, via electrodes, to cause the tongue to move and to alleviate the OSA episode.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of detecting obstructive sleep apnea (OSA) by a user, the method comprising:
receiving audio data emanating from the user while the user is sleeping, the audio data comprising multiple distinct frequency bands;
analyzing the audio data to detect patterns in frequency content that matches two or more formants of a plurality of formants, the two or more formants indicating a position of a tongue of the user;
based on the analyzing, determining when the tongue has moved from a forward position to a rear position in an oral cavity of the user; and
generating a signal indicating an OSA episode when it is determined that the tongue has moved from a forward position to a rear position, the generating further comprising using a first machine learning model that is trained on a pattern of F1/F2 evolution over time.

2. The method of claim 1, further comprising:
initiating an electrical stimulation of nerves of the user, via electrodes, to cause the tongue to move and to alleviate the OSA episode.

3. The method of claim 1, the analyzing comprising:
converting a time domain audio signal into a frequency domain;
creating a data sequence to show, at each sampled time step, an energy content of the audio at each specified frequency band.

4. The method of claim 1, further comprising adjusting the frequency bands while the user is sleeping in response to changes in breathing of the user in response to at least one of a change in a sleeping position or a congestion in a passageway.

5. The method of claim 1, further comprising measuring a duration of each inspiration of the user to estimate a magnitude of an indrawn breath.

6. The method of claim 1, the two or more formants comprising an F1 formant and an F2 formant.

7. The method of claim 6, further comprising:
training a machine learning model based on formant data over time;
using the trained machine learning model for the analyzing.

8. The method of claim 2, further comprising using a trained machine learning model that was trained based on formant data or breathing data over time to adjust the electrical stimulation over time as the patterns of formants or breathing changes.

9. An obstructive sleep apnea (OSA) detection system comprising:
a respiration monitoring device adapted to receiving audio data emanating from the user while the user is sleeping, the audio data comprising multiple distinct frequency bands; and
one or more processors adapted to analyze the audio data to detect patterns in frequency content that matches two or more formants of a plurality of formants, the two or more formants indicating a position of a tongue of the user, based on the analyzing, determine when the tongue has moved from a forward position to a rear position in an oral cavity of the user, and generate a signal indicating an OSA episode when it is determined that the tongue has moved from a forward position to a rear position, the generate further comprising using a first machine learning model that is trained on a pattern of F1/F2 evolution over time.

10. The OSA detection system of claim 9, further comprising:
a patch adapted to receive the signal and, in response, initiate an electrical stimulation of nerves of the user, via electrodes, to cause the tongue to move and to alleviate the OSA episode.

11. The OSA detection system of claim 9, the analyzing comprising:
converting a time domain audio signal into a frequency domain;
creating a data sequence to show, at each sampled time step, an energy content of the audio at each specified frequency band.

12. The OSA detection system of claim 9, the one or more processors further comprising adjusting the frequency bands while the user is sleeping in response to changes in breathing of the user in response to at least one of a change in a sleeping position or a congestion in a passageway.

13. The OSA detection system of claim 9, the one or more processors further comprising measuring a duration of each inspiration of the user to estimate a magnitude of an indrawn breath.

14. The OSA detection system of claim 9, the two or more formants comprising an F1 formant and an F2 formant.

15. The OSA detection system of claim 9, further comprising:
a machine learning model that is trained based on formant data over time, the one or more processors using the trained machine learning model for the analyzing.

16. The OSA detection system of claim 10, the one or more processors further comprising using a trained machine learning model that was trained based on formant data or breathing data over time to adjust the electrical stimulation over time as the patterns of formants or breathing changes.

17. A non-transitory computer-readable medium storing instructions which, when executed by at least one of a plurality of processors, cause the processor to detect obstructive sleep apnea (OSA) by a user, the detecting comprising:
receiving audio data emanating from the user while the user is sleeping, the audio data comprising multiple distinct frequency bands;
analyzing the audio data to detect patterns in frequency content that matches two or more formants of a plurality of formants, the two or more formants indicating a position of a tongue of the user;
based on the analyzing, determining when the tongue has moved from a forward position to a rear position in an oral cavity of the user; and
generating a signal indicating an OSA episode when it is determined that the tongue has moved from a forward position to a rear position, the generating further comprising using a first machine learning model that is trained on a pattern of F1/F2 evolution over time.

18. The non-transitory computer-readable medium of claim 17, the detecting further comprising:
initiating an electrical stimulation of nerves of the user, via electrodes, to cause the tongue to move and to alleviate the OSA episode.

19. The non-transitory computer-readable medium of claim 17, the analyzing comprising:
converting a time domain audio signal into a frequency domain;
creating a data sequence to show, at each sampled time step, an energy content of the audio at each specified frequency band.

20. The non-transitory computer-readable medium of claim 17, the detecting further comprising adjusting the frequency bands while the user is sleeping in response to changes in breathing of the user in response to at least one of a change in a sleeping position or a congestion in a passageway.
